(19) United States
(12) Patent Application Publication
(10) Pub. No.: US 2024/0331821 A1
(43) Pub. Date: Oct. 3, 2024

(54) MEDICAL CONVERSATIONAL INTELLIGENCE

(71) Applicant: Amazon Technologies, Inc., Seattle, WA (US)

(72) Inventors: Vijit Gupta, Mercer Island, WA (US); Matthew Chih-Hui Chiou, Seattle, WA (US); Amiya Kishor Chakraborty, Seattle, WA (US); Anuroop Arora, Seattle, WA (US); Varun Sembium Varadarajan, Redmond, WA (US); Sarthak Handa, Seattle, WA (US); Amit Vithal Sawant, New Brunswick, NJ (US); Glen Herschel Carpenter, Arvada, CO (US); Jesse Deng, Seattle, WA (US); Mohit Narendra Gupta, Seattle, WA (US); Rohil Bhattarai, Seattle, WA (US); Samuel Benjamin Schiff, New York, NY (US); Shane Michael McGookey, Seattle, WA (US); Tianze Zhang, Long Island City, NY (US)

(73) Assignee: Amazon Technologies, Inc., Seattle, WA (US)

(21) Appl. No.: 18/194,350

(22) Filed: Mar. 31, 2023

(51) Int. Cl. G16H 15/00; G06F 40/40; G16H 10/60

(52) U.S. Cl. CPC G16H 15/00; G06F 40/40; G16H 10/60

(57) ABSTRACT

Systems and methods for performing medical audio summarizing for medical conversations are disclosed. An audio file and meta data for a medical conversation are provided to a medical audio summarization system. A transcription machine learning model is used by the medical audio summarization system to generate a transcript and a natural language processing service of the medical audio summarization system is used to generate a summary of the transcript. The natural language processing service may include at least four machine learning models that identify medical entities in the transcript, identify speaker roles in the transcript, determine sections of the transcript corresponding to the summary, and extract or abstract phrases for the summary. The identified medical entities and speaker roles, determined sections, and extracted or abstracted phrases may then be used to generate the summary.

### BACKGROUND

Some enterprises implement services for generating transcripts of conversations. For example, automatic speech recognition (ASR) may be used to generate transcripts. Also, some enterprises provide natural language processing (NLP) services. However, general purpose ASR and NLP systems may not function well for medical conversations. For example, due to specialized terms used in the medical industry. Also, NLP services may provide acceptable results when asked to perform discrete low-level tasks, but may provide low quality results when asked to perform higher-level tasks such as generating an overall summary of a medical conversation.

### BRIEF DESCRIPTION OF THE DRAWINGS

FIG. 1A is a block diagram illustrating a medical audio summarization service, wherein a medical transcription service generates a transcript of a medical conversation based on audio and meta data from the medical conversation and a medical natural language processing engine generates a summary document based on the transcript of the medical conversation using a plurality of different machine learning models, according to some embodiments.

FIG. 1B is a block diagram illustrating a summarization module, wherein a sectioning model, an extraction model, an abstraction model, and an evidence linkage model are used as part of a process to generate a summary document of a medical conversation, in some embodiments.

FIG. 2 illustrates an example provider network that may implement a medical audio summarization service that implements a medical transcription engine and a medical natural language processing engine, according to some embodiments.

FIG. 3 is a flow diagram illustrating a process of generating a transcript of a medical conversation via a transcription model trained using medical terminology and generating a summary of the transcript using a medical natural processing service, according to some embodiments.

FIG. 4 is a block diagram illustrating medical entities in a transcript of a medical conversation, a workflow for generating a summary of the medical conversation, and a process of detecting medical entities in the transcript of the medical conversation, according to some embodiments.

FIG. 5 is a block diagram illustrating speaker roles in a transcript of a medical conversation, a workflow for generating a summary of the medical conversation, and a process of identifying speaker roles in the transcript of the medical conversation, according to some embodiments.

FIG. 6 is a block diagram illustrating labeled sections in a transcript of a medical conversation, a workflow for generating a summary of the medical conversation, and a process of identifying sections for a summary in a transcript of the medical conversation, according to some embodiments.

FIG. 7 is a block diagram illustrating extracted phrases in a transcript of a medical conversation, a workflow for generating a summary of the medical conversation, and a process of extracting and/or abstracting phrases from a transcript of the medical conversation, according to some embodiments.

FIG. 8 is a block diagram illustrating an annotated transcript of a medical conversation indicating identified medical entities, identified speaker roles, labeled sections, and extracted phrases of the transcript and a generated summary of the medical conversation that has been generated based on the identified medical entities, the identified speaker roles, the determined sections, and the extracted phrases from the transcript, according to some embodiments.

FIG. 9 is a block diagram illustrating four machine learning models used for generating a summary of a medical conversation, wherein respective ones of the models are trained to receive, as input, a transcript provided by a preceding one of the models and wherein the models are trained using an annotated version of the transcript provided by the preceding model, according to some embodiments.

FIG. 10 is a flow diagram illustrating a process of generating a transcript of a medical conversation and generating a summary document of the medical conversation based on the transcript, using four machine learning models trained as described in FIG. 9, according to some embodiments.

FIG. 11 is a block diagram illustrating an example computer system that implements portions of the medical audio summarizing described herein, according to some embodiments.

While embodiments are described herein by way of example for several embodiments and illustrative drawings, those skilled in the art will recognize that embodiments are not limited to the embodiments or drawings described. The drawings and detailed description thereto are not intended to limit embodiments to the particular form disclosed, but on the contrary, the intention is to cover all modifications, equivalents and alternatives falling within the spirit and scope as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including, but not limited to.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

“Comprising.” This term is open-ended. As used in the claims, this term does not foreclose additional structure or steps. Consider a claim that recites: “An apparatus comprising one or more processor units . . . .” Such a claim does not foreclose the apparatus from including additional components.

“Configured To.” Various units, circuits, or other components may be described or claimed as “configured to” perform a task or tasks. In such contexts, “configured to” is used to connote structure by indicating that the units/components include structure that performs those task or tasks during operation. As such, the unit/component can be said to be configured to perform the task even when the specified unit/component is not currently operational (e.g., is not on). The units/components used with the “configured to” language include hardware—for example, circuits, memory storing program instructions executable to implement the operation, etc. Reciting that a unit/component is “configured to” perform one or more tasks is expressly intended not to invoke 35 U.S.C. § 112, paragraph (f), for that unit/component. Additionally, “configured to” can include generic structure that is manipulated by software or firmware to operate in manner that is capable of performing the task(s) at issue.

“Based On” or “Dependent On.” As used herein, these terms are used to describe one or more factors that affect a determination. These terms do not foreclose additional factors that may affect a determination. That is, a determination may be solely based on those factors or based, at least in part, on those factors. Consider the phrase “determine A based on B.” While in this case, B is a factor that affects the determination of A, such a phrase does not foreclose the determination of A from also being based on C. In other instances, A may be determined based solely on B.

“Or.” When used in the claims, the term “or” is used as an inclusive or and not as an exclusive or. For example, the phrase “at least one of x, y, or z” means any one of x, y, and z, as well as any combination thereof.

It will also be understood that, although the terms 1, 2, N, etc. may be used herein to describe various elements, these elements should not be limited by these terms. These terms are only used to distinguish one element from another. For example, a component with the term 1 could be termed a second component, and, similarly, a component with the term 2 could be termed a first component, without departing from the scope of the present invention. The first components and the second component are both components, but they are not the same components. Also, the term N indicates that an Nth amount of the elements may or may not exist depending on the embodiments.

### DETAILED DESCRIPTION OF EMBODIMENTS

The burden of documenting clinical visits is one of the largest sources of inefficiency in healthcare. Physicians often spend considerable time navigating different tabs, fields, and drop-downs in electronic health record (EHR) systems to capture details such as medications, allergies, and medical conditions. Physicians also make short-hand notes during the consultation on topics such as patient's history of illness or clinical assessment, and enter their summarized notes in the EHR systems after the visit, often during the off-peak hours. Even with the help of scribes, creating clinical documentation and summaries can be time consuming and inefficient. Training a machine learning model for generating transcripts and summaries of medical conversations require a range of resources which adds cost and complexity.

Additionally, current machine learning models are not well suited to the nuanced tasks of generating summaries of medical conversations. For example, the large number of variables involved in generating a summary of a medical conversation and the specialized terms used in the medical industry may cause inaccurate results when using current machine learning models. Also, due to the importance of accuracy in medical records, a very low (or zero) error rate may be required in medical summaries. For example, mis-stating a drug dosage in a summary and using such information subsequently may lead to poor patient outcomes. Thus, a highly accurate medical audio summarization service is needed.

To address these issues and/or other issues, in some embodiments, a system may provide a HIPAA-eligible conversational intelligence capability trained to understand patient-physician conversations across diverse medical specialties. In order to overcome accuracy problems of current machine learning models, a medical transcription engine may be trained using medical training data that includes annotated medial entities that the medical transcription engine is to be trained to detect. Also, a medical natural language processing engine may be trained with annotated versions of medical transcripts generated by the medical transcription engine. Additionally, instead of using a single (or shared) machine learning model to perform various tasks involved in summary generation from a transcript, different tasks involved in generating a summary may be separated out into discrete tasks of a workflow performed by a medical natural language processing engine. Additionally, discrete machine learning models may be trained to perform respective ones of the discrete tasks of the workflow, wherein the discrete machine learning models are trained to perform a narrow task and are also trained using specialized training data (that has been annotated), wherein the specialized training data is based on outputs generated by a preceding task in the workflow, wherein the preceding task uses its own discrete machine learning model specially trained to perform a narrow task involved in the preceding task in the workflow. In this way, highly accurate medical transcripts may be generated with a high-level of confidence. For example, a single machine learning model that has been trained to go directly from an input transcript to an output summary may make errors in the summary, such as attributing phrases spoken by a patient to be marked as if they were spoken by a physician, incorrectly stating a drug name or dosage, etc. However, when separated into discrete tasks and integrated together via shared data that builds on a preceding task, a medical natural language processing engine comprising multiple specially trained machine learning models may be configured to identify phrase attribution with very low error, and identify medical entities, such as drug names and dosages, with high accuracy, as well as perform other summarization tasks with a high-level of accuracy and confidence.

In some embodiments, a medical audio summarization service may receive a request to generate a transcript and a summary of a medical conversation, with a medical conversation job packet to be summarized, including audio data and meta data of the medical conversation. In some embodiments, the transcript may be generated via a medical transcription service based on the audio data from the medical conversation job packet. In some embodiments, the transcript may be generated while the medical conversation is occurring. The transcription service may receive audio data from the medical conversation and may begin to generate the transcript while continuing to receive audio data from the same medical conversation. The transcript and meta data may be provided to a medical natural language processing service to generate a summary of the medical conversation using the transcript.

To generate the summary of the medical conversation, a plurality of specialized machine learning models may be implemented to perform discrete tasks, such as identify medical entities and speaker roles in the transcript, determine sections of the transcript corresponding to the summary, extract phrases for the summary, and/or abstract phrases for the summary. In some embodiments, medical entities including but not limited to medical terms for medicines and diseases may be identified in the transcript using a first machine learning model. Using a second machine learning model, speaker roles, such as physician and patient, may be identified. Portions of the transcript that correspond to subject matter of sections for the summary may be determined using a third machine learning model. The third machine learning model or a fourth machine learning model may be implemented to extract phrases from the sections for the summary or to abstract phrases for the summary. In some embodiments, the abstracting phrases may be performed by paraphrasing from the sections of the transcript. The summary may then be generated using the identified medical entities, identified speaker roles, determined sections, and extracted/abstracted phrases.

In some embodiments, the respective machine learning models may be used in different orders, but may be trained in whichever order the machine learning models are to be used. For example, in some embodiments, speaker role identification may be performed before medical entity identification, but in such a case, the medical entity identification model may be trained using training data this is output from the speaker role identification task. In other embodiments, medical entity identification may be performed prior to speaker role identification, in which case the speaker role identification model may be trained using training data that is output from the medical entity identification task.

In some embodiments, a notification report indicating the generation of the summary document may be provided. An application programmatic interface (API) may be implemented for providing the summary for upload to an electronic health record service.

In some embodiments, the transcript may be merged with the results of a preceding model before being used for a future model. For example, a merged transcript including identified medical entities may be the transcript used for identifying the speaker roles with the second machine learning model. In some embodiments, the machine learning models may be trained based on merged transcripts comprising results from previously trained machine learning models. For example, if the first and second machine learning models are trained before the third machine learning model, third machine learning model may be trained with a transcript including identified medical entities and speaker roles.

In some embodiments, customer preferences may be uploaded to a customer interface to update the machine learning model of the transcription service and the machine learning models for generating the summary. For example, a physician may require a specific summary template and may upload the template to the customer interface to further train the machine learning models. In another example, a physician may upload their own training data including annotated transcripts for training the machine learning models. For example, physicians practicing in different specialized fields may desire to have models trained using terms specific to their specialties. In some embodiments, a medical audio summarization service may maintain specialty specific models regardless of whether or not a physician provides practice specific training data.

As will be appreciated by those skilled in the art, features of the system disclosed herein may be implemented in computer systems to solve technical problems in the state of the art and to improve the functioning of the computer systems. For example, as discussed above, and as discussed in more detail below, such features of the system improve medical conversation transcript and summary generating in a way that provides higher accuracy than prior approaches. This may be achieved, at least in part, by dividing the summarization process into discrete tasks and training discrete machine learning models in an integrated fashion to perform the respective discrete tasks. Such features also improve the functioning of the computer system by requiring less computational resources than conventional machine learning models. For example, computational resources required to perform the discrete tasks may sum to be significantly less than the computational resources required to perform summarization using a single model with significantly more variables. These and other features and advantages of the disclosed system are discussed in further detail below, in connection with the figures.

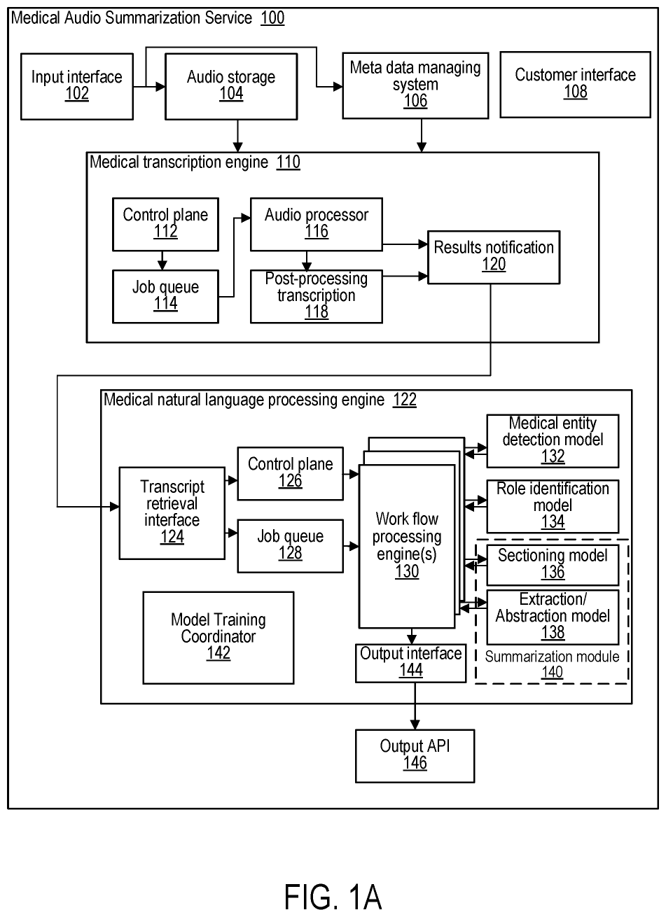

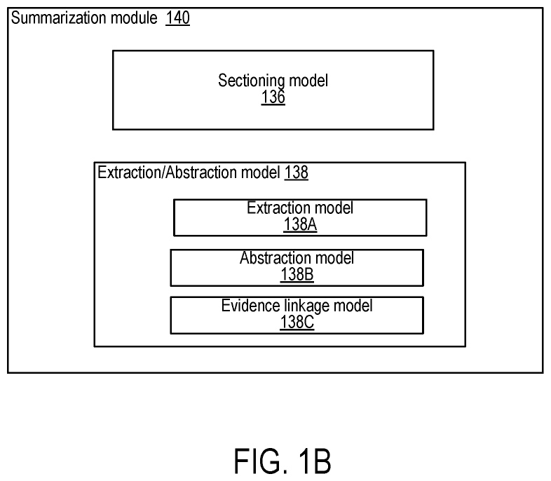

FIG. 1A is a block diagram illustrating a medical audio summarization service, wherein a medical transcription service generates a transcript of a medical conversation based on audio and meta data from the medical conversation and a medical natural language processing engine generates a summary document based on the transcript of the medical conversation using machine learning models, according to some embodiments.

In some embodiments, medical audio summarization is performed, such as by a medical audio summarization service 100, and may resemble embodiments as shown in FIG. 1A. In some embodiments, an input interface 102 may receive an audio file including meta data of a medical conversation. For example, a physician may upload a clinical visit audio between a patient and the physician to the input interface 102 in order to generate a transcript and a summary. The input interface 102 may then provide the audio file to an audio storage 104 and also provide the meta data for the audio file to a meta data managing system 106. Once the medical transcription engine 110 receives a job request, the engine may access the audio file and the meta data of the medical conversation, from the audio storage 104 and the meta data managing system 106, respectively. A control plane 112 may send the job request to be queued to a job queue 114. The audio processor 116 may then process the job request from the job queue 114 and generate the transcript of the medical conversation. In some embodiments, the results may be provided to the post-processing transcription 118 for post-processing before providing a results notification 120. The results notification 120 indicating generation of the transcript may be provided to the medical natural language processing engine 122. In some embodiments, a first amount of audio data of the medical conversation may be provided to the medical transcription engine 110, which the patient and physician are still talking and generating a second (or subsequent) amount of the audio data. The transcript may be generated based on the first amount of audio data and the medical transcription engine 110 may receive the second amount of the audio data while continuing to generate the transcript. For example, the transcript may be generated during a clinical visit.

In some embodiments, a medical natural language processing engine 122 may receive notification of a job request to generate a summary and may also receive the transcript needed for the job request via a transcript retrieval interface 124. Notification of the job request and the transcript may be provided to a control plane 126 for the medical natural language processing engine 122 and the job request and transcript may be provided to a job queue 128. A work flow processing engine 130 may be instantiated by the control plane 126 and may receive the job request and the transcript from the job queue 128. The work flow processing engine 130 may then invoke machine learning models such as a medical entity detection model 132 to identify medical entities, a role identification model 134 to identify speaker roles, and a summarization module 140 including a sectioning model 136 to determine sections for the summary, and an extraction/abstraction model 138 to extract or abstract phrases for the summary. The work flow processing engine 130 may then generate the summary based on the results from the invocation of the machine learning models. For example, a computing instance instantiated as a workflow processing engine 130 may access respective ones of the models to perform discrete tasks, such as medical entity detection, role identification, sectioning, extraction, and abstraction. The workflow processing engine 130 may merge results from each task into a current version of the transcript that is being updated as the discrete tasks are performed. The currently updated (and merged) version of the transcript may be used as an input to perform respective ones of the subsequent discrete tasks.

For example, in some embodiments, the work flow processing engine 130 may merge the results from a task performed using a prior model with the transcript and use the merged transcript to determine results for a task that uses the next model. For example, a workflow worker instance of the work flow processing engine 130 may invoke a medical entity detection model 132 to identify medical entities in a transcript. The results may then be merged with the transcript to include the original transcript with the identified medical entities. The workflow worker instance may then invoke the role identification model 134 to identify speaker roles in the merged transcript. The identified speaker role results may then be merged with the merged transcript to include the identified medical entities and identified speaker roles. The workflow worker instance may invoke the section model 136 to determine portions of the merged transcript corresponding to subject matter of sections for the summary document. The section results may then be merged with the merged transcript to include the identified medical entities, the identified speaker roles, and the determined sections. The workflow worker instance may invoke the extraction/abstraction model 138 to extract and/or abstract phrases in the merged transcript for the summary.

In some embodiments, the models may be invoked in a different order. For example, the role identification model 134 may be invoked first causing the identified speaker role results to be merged with the transcript. The medical entity detection model 132 may then be performed meaning the workflow worker instance may use the transcript merged with the identified speaker role results to determine the medical entities in the merged transcript. In some embodiments, the information gained from use of the previous model may be used for the next model. For example, if the role identification model 134 is performed using a merged transcript including identified medical entities, the role identification model 134 may identify that the physician as the speaker who used the most technical medical entities.

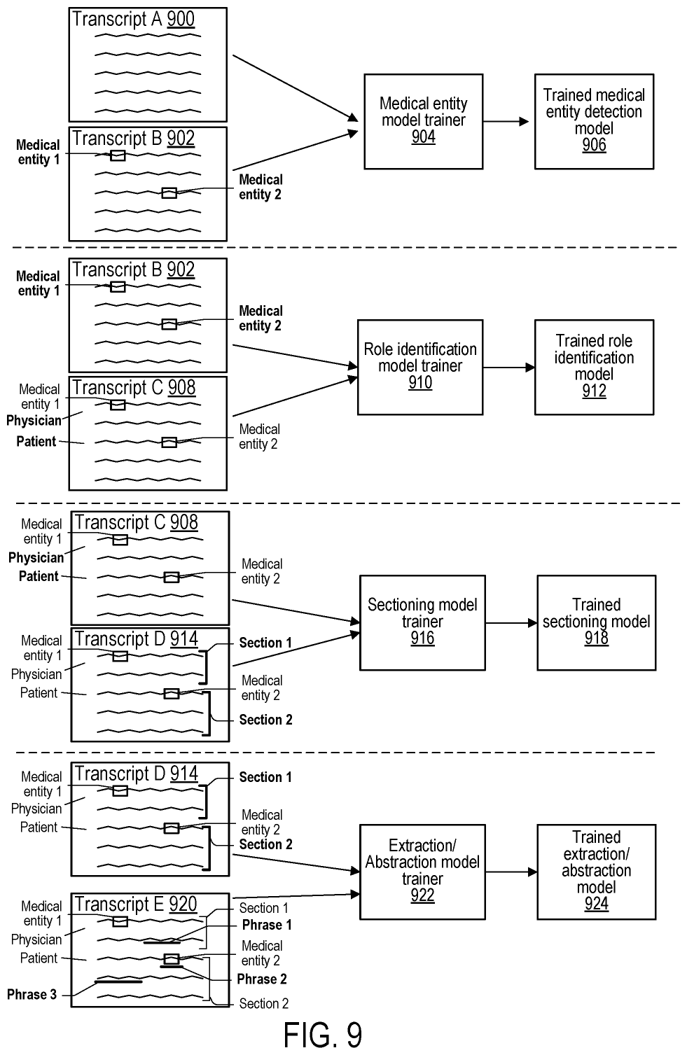

In some embodiments, a model training coordinator 142 may be used for training the machine learning models with labeled training data, such as annotated transcripts. For example, training is further discussed in detail in regard to FIG. 9. In some embodiments, the model training coordinator 142 may be used offline. In some embodiments, a customer interface 108 may receive customer preferences and/or training data. For example, a customer may upload a preferred summary template for the generated summary to be based on. In another example, in a case where the physician is a specialist in a specific field, the physician may want to train the medical transcription engine 110 and/or the medical natural language processing engine 122 with their own labeled training data. In such a case, the specialist may upload transcripts from their specific field to tailor the engine(s) to the specific field. The newly updated medical transcription engine 110 and the newly updated medical natural language processing engine 122 may better recognize specific terms from the specific field and improve the quality of a generated transcript and generated summary for the specialist.

Once the summary is generated, the work flow processing engine 130 may provide the generated summary to an output interface 144. The output interface 144 may notify the customer of the completed job request. In some embodiments, the output interface may provide a notification of a completed job to the output API 146. In some embodiments, the output API 146 may be implemented to provide the summary for upload to an electronic health record or may push the summary out to an electronic health record, in response to a notification of a completed job.

FIG. 1B is a block diagram illustrating a summarization module, wherein a sectioning model, an extraction model, an abstraction model, and an evidence linkage model are used as part of a process to generate a summary document of a medical conversation, in some embodiments.

Some embodiments, such as shown in FIG. 1A may include further features such as shown in FIG. 1B. In some embodiments, the summarization module 140 may include the sectioning model 136 and the extraction/abstraction model 138 as shown in FIG. 1A. In some embodiments, the extraction/abstraction model 138 may include an extraction model 138A, an abstraction model 138B, and an evidence linkage model 138C. In some embodiments, the extraction/abstraction model 138 may include three models as shown in FIG. 1B or in some embodiments, one or more of the models may be omitted or combined. For example, in some embodiments, an evidence linkage task may be omitted if phrase extraction is used, because the source of the phrases in the summary is obvious (e.g., extracted words in the summary can be matched to words in the transcript). In some embodiments, an extraction task may be omitted and the abstracted representation of a section may be generated from a corresponding section without necessarily performing a separate extraction task. In such embodiments, a further task of evidence linkage may be performed using the evidence linkage model 138C, for example in order to show a link between content in the summary and the source for generating such content in the transcript.

The extraction model 138A may be a machine learning model used to extract phrases from the transcript for the summary. The abstraction model 138B may be a machine learning model utilized to abstract phrases or sections from the transcript for the summary. In some embodiments, the extraction model 138A may be used first and the abstraction model 138B may be utilized with results from the extraction model 138A to abstract the extracted phrases. In some embodiments, the abstraction model 138B may be utilized without the results from the extraction model 138A and may analyze the sections of the transcript to paraphrase sections for the summary. The evidence linkage model 138C may be a generative model used to link either the extracted or abstracted phrases from the generated summary to the transcript based on confidence values. The higher the confidence value the stronger likelihood that the extracted or abstracted phrase from the generated summary is related to a specific portion of the transcript. Thus, the evidence linkage model 138C may be used to ensure the quality of the generated summary by requiring the confidence values to be higher than a determined threshold.

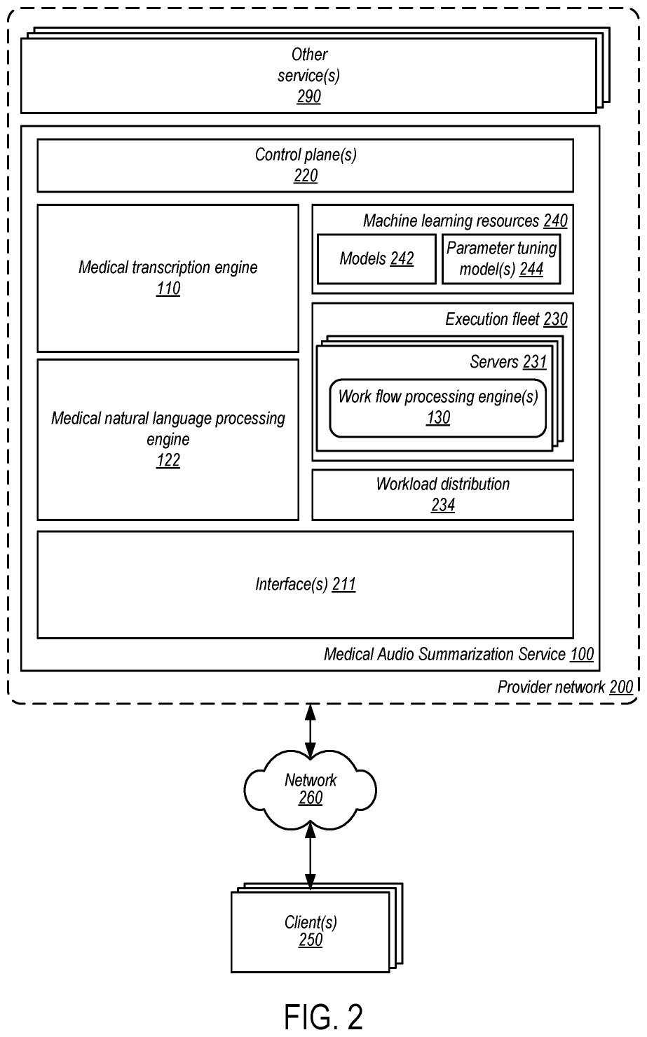

FIG. 2 illustrates an example provider network that may implement a medical audio summarization service that implements a medical transcription engine and a medical natural language processing engine, according to some embodiments.

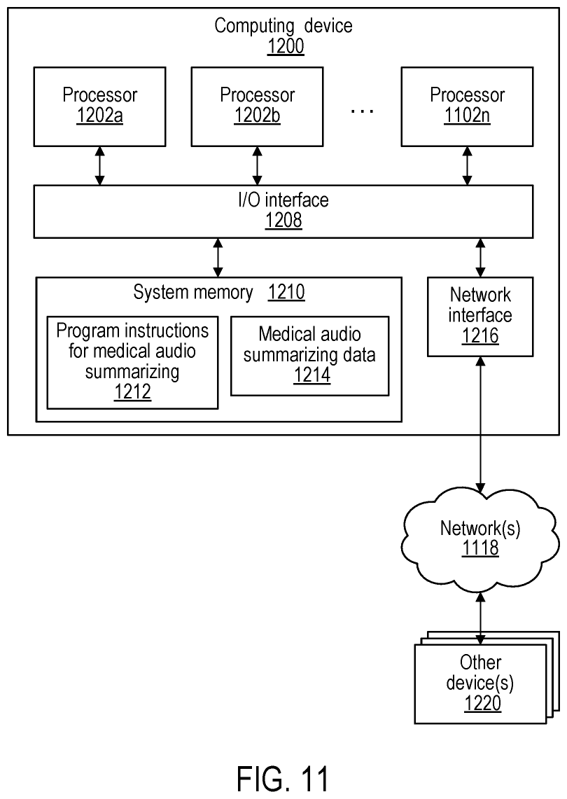

Some embodiments, such as shown in FIGS. 1A-B, may include further features such as shown in FIG. 2. A provider network 200 may be a private or closed system or may be set up by an entity such as a company or a public sector organization to provide one or more services (such as various types of cloud-based storage) accessible via the Internet and/or other networks to clients 250, in some embodiments. The provider network 200 may be implemented in a single location or may include numerous data centers hosting various resource pools, such as collections of physical and/or virtualized computer servers, storage devices, networking equipment and the like (e.g., computing system 1100 described below with regard to FIG. 11), needed to implement and distribute the infrastructure and services offered by the provider network 200, in some embodiments. For example, the provider network 200 may implement various computing resources or services, such as a medical audio summarization service 100, and/or any other type of network-based services 290 (which may include a virtual compute service and various other types of storage, database or data processing, analysis, communication, event handling, visualization, data cataloging, data ingestion (e.g., ETL), and security services), in some embodiments.

In various embodiments, the medical audio summarization service 100 may implement interface(s) 211 to allow clients (e.g., client(s) 250 or clients implemented internally within provider network 200, such as a client application hosted on another provider network service like an event driven code execution service or virtual compute service) to interact with the medical audio summarization service 100. The interface(s) 211 may be one or more of graphical user interfaces, programmatic interfaces that implement Application Program Interfaces (APIs) and/or command line interfaces, such as input interface 102, customer interface 108, and/or output interface 144, for example as shown in FIG. 1A.

In at least some embodiments, workflow processing engine(s) 130 may be implemented on servers 231 to initiate tasks for a medical transcription engine 110 and a medical natural processing engine 122. The workload distribution 234, comprising one or more computing devices, may be responsible for selecting the particular server 231 in execution fleet 230 that is to be used to implement a workflow engine to be used to perform a given job. The medical audio summarization service 100 may implement control plane(s) 220 to perform various control operations to implement the features of medical audio summarization service 100, such as control plane 112 and control plane 126 in FIG. 1A. For example, the control plane(s) 220 may monitor the health and performance of requests at different components, such as workload distribution 234, servers 231, machine learning resources 240, the medical transcription engine 110, and the medical natural language processing engine 122. The control plane 220 may, in some embodiments, arbitrate, balance, select, or dispatch requests to different components in various embodiments.

The medical audio summarization service 100 may utilize machine learning resources 240. The machine learning resources 240 may include parameter tuning model 244 and models 242 such as the medical entity detection model 132, the role identification model 134, the sectioning model 136, and the extraction/abstraction model 138, for example as shown in FIG. 1A. In some embodiments, one or more machine learning-based parameter tuning models 244 may be used to adjust the models during training as well as to analyze input received from a customer, such as from the customer interface 108 shown in FIG. 1A, and adjust one or more of the models 242.

Generally speaking, clients 250 may encompass any type of client that can submit network-based requests to provider network 200 via network 260, including requests for the medical audio summarization service 100 (e.g., a request to generate a transcript and summary of a medical conversation). For example, a given client 250 may include a suitable version of a web browser, or may include a plug-in module or other type of code module that can execute as an extension to or within an execution environment provided by a web browser.

In some embodiments, a client 250 may provide access to provider network 200 to other applications in a manner that is transparent to those applications. Clients 250 may convey network-based services requests (e.g., requests to interact with services like medical audio summarization service 100) via network 260, in some embodiments. In various embodiments, network 260 may encompass any suitable combination of networking hardware and protocols necessary to establish network-based-based communications between clients 250 and provider network 200. For example, network 260 may generally encompass the various telecommunications networks and service providers that collectively implement the Internet. Network 260 may also include private networks such as local area networks (LANs) or wide area networks (WANs) as well as public or private wireless networks, in one embodiment. For example, both a given client 250 and provider network 200 may be respectively provisioned within enterprises having their own internal networks. In such an embodiment, network 260 may include the hardware (e.g., modems, routers, switches, load balancers, proxy servers, etc.) and software (e.g., protocol stacks, accounting software, firewall/security software, etc.) necessary to establish a networking link between the given client 250 and the Internet as well as between the Internet and provider network 200. It is noted that in some embodiments, clients 250 may communicate with provider network 200 using a private network rather than the public Internet.

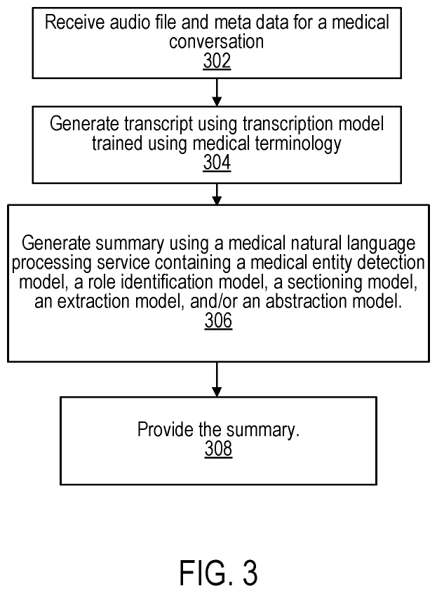

FIG. 3 is a flow diagram illustrating a process of generating a transcript of a medical conversation via a transcription model trained using medical terminology and generating a summary of the transcript using a medical natural processing service, according to some embodiments.

In some embodiments, a process for generating a transcript and a summary of a medical conversation may resemble a process such as that which is shown in FIG. 3. In block 302, an audio file and meta data for a medical conversation may be received. In block 304, a transcript may be generated using a transcription model, such as the medical transcription engine 110 shown in FIGS. 1-2, trained using medical terminology. For example, the transcription model may be trained with audio files and transcripts of clinical visits between a physician and patient. In block 306, a summary may be generated using a medical natural language processing service containing a medical entity detection model, a role identification model, a sectioning model, an extraction model, and/or an abstraction model, such as the medical natural language processing engine 122 in FIGS. 1-2, that have been trained via a process as shown in FIG. 9. In block 308, the summary may be provided. For example, the summary may be provided to an output interface or API for a customer.

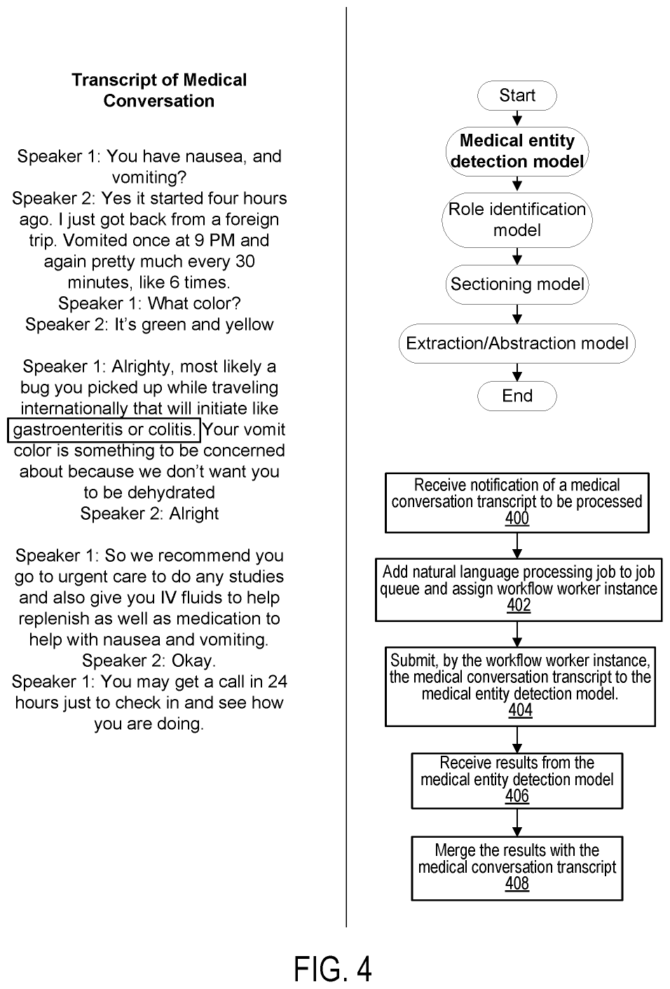

FIG. 4 is a block diagram illustrating medical entities in a transcript of a medical conversation, a workflow for generating a summary of the medical conversation, and a process of detecting medical entities in the transcript of the medical conversation, according to some embodiments.

Some embodiments, such as shown in FIGS. 1A, 1B, 2 and 3, may include further features, such as shown in FIG. 4. In some embodiments, a workflow for generating a summary of a transcript for a medical conversation may involve machine learning models including a medical entity detection model, a role identification model, a sectioning model, and an extraction/abstraction model. An engine, such as the work flow processing engine 130 from FIG. 1A, may utilize the medical entity detection model to analyze the transcript of the medical conversation and detect medical terms such as gastroenteritis or colitis as shown in the transcript of the medical conversation in FIG. 4 identified by a box around the terms. Medical terms may include but are not limited to medicines, disease names, or scientific phrases used in the medical field.

In block 400, a notification of a medical conversation transcript to be processed may be received. For example, the transcript retrieval interface 124 shown in FIG. 1A may receive such a notification. In block 402, a natural language processing job may be added to a job queue, such as the job queue 128 in FIG. 1A, and a workflow worker instance may be assigned for the job, such as the work flow processing engine 130 in FIG. 1A. In block 404, the medical conversation transcript may be submitted by the workflow worker instance to the medical entity detection model. In block 406, the results from the medical entity detection model may be determined. An example of results includes the identified medical entities “gastroenteritis or colitis” in the transcript of the medical conversation shown in FIG. 4. In block 408, the results may be merged with the medical conversation transcript so the merged transcript includes the original transcript and the identified medical entities.

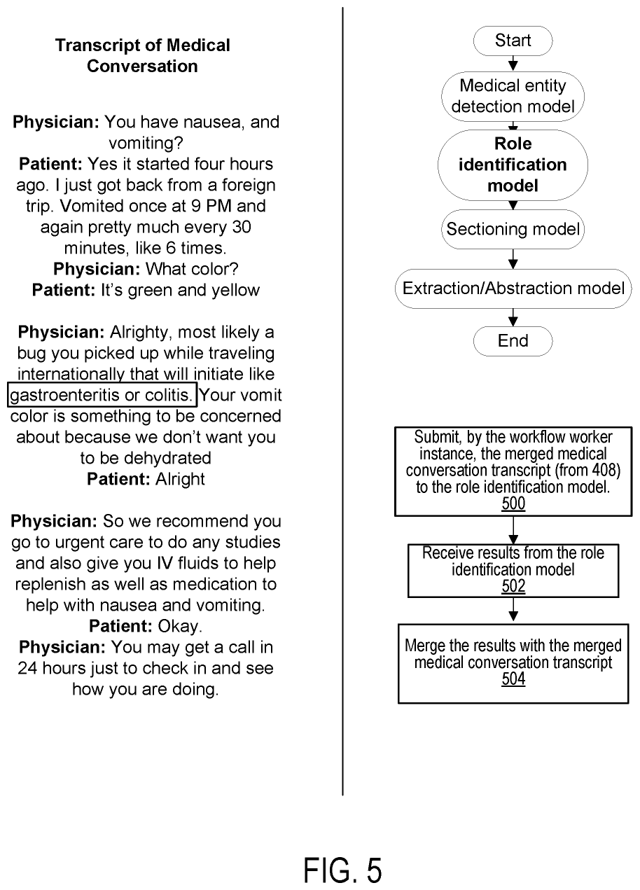

FIG. 5 is a block diagram illustrating speaker roles in a transcript of a medical conversation, a workflow for generating a summary of the medical conversation, and a process of identifying speaker roles in the transcript of the medical conversation, according to some embodiments

Some embodiments, such as shown in FIGS. 1A, 1B, 2, 3, and 4 may include further features, such as shown in FIG. 5. In some embodiments, a workflow for generating a summary of a transcript for a medical conversation may involve machine learning models including a medical entity detection model, a role identification model, a sectioning model, and an extraction/abstraction model. An engine, such as the work flow processing engine 130 from FIG. 1A, may utilize the role identification model to analyze the transcript of the medical conversation and identify the speakers in a transcript as a physician or patient as shown in the merged transcript of the medical conversation in FIG. 5. The speaker roles may be identified by examining the sentences of each speaker. In some embodiments, a merged transcript including results from a preceding model may be used to aide the role identification model in determining the speaker roles. For example, the role identification model may have been in trained in a way that assumes a physician would state more medical entities than a patient and may use this training to determine which speaker is the physician.

In block 500, the merged medical conversation transcript (from block 408) may be submitted by the workflow worker instance to the role identification model. In block 502, the results from the role identification model may be received. An example of results includes the identified physician and patient roles bolded in the transcript of the medical conversation shown in FIG. 5. In block 504, the results may be merged with the merged medical conversation transcript so the merged transcript includes the original transcript, the identified medical entities, and the identified speaker roles.

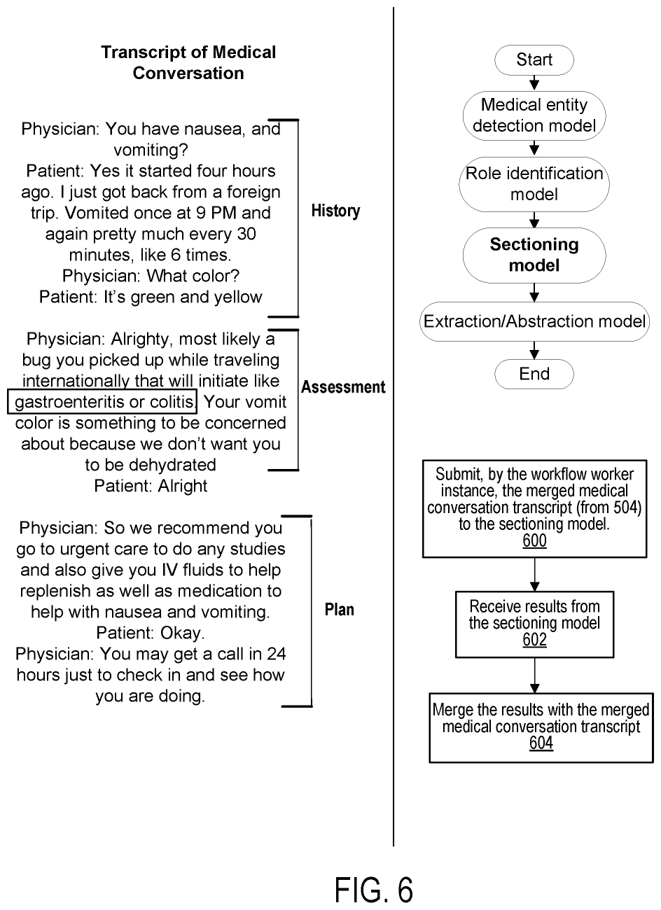

FIG. 6 is a block diagram illustrating labeled sections in a transcript of a medical conversation, a workflow for generating a summary of the medical conversation, and a process of identifying sections for a summary in a transcript of the medical conversation, according to some embodiments.

Some embodiments, such as shown in FIGS. 1A, 1B, 2, 3, 4, and 5 may include further features, such as shown in FIG. 6. In some embodiments, a workflow for generating a summary of a transcript for a medical conversation may involve machine learning models including a medical entity detection model, a role identification model, a sectioning model, and an extraction/abstraction model. An engine, such as the work flow processing engine 130 from FIG. 1A, may utilize the sectioning model to analyze the transcript of the medical conversation and determine portions of the transcript corresponding to sections for a summary as shown in the merged transcript of the medical conversation in FIG. 6 such as “History”, “Assessment”, and “Plan”. In some embodiments, such as in FIG. 6, the “History” section may represent sentences discussing a medical history of the patient and any important background information for the patient. The “Assessment” section may represent sentences discussing the physician's observations and diagnosis of the patient. The “Plan” section may represent sentences discussing the physician's instructions and recommendations for the patient.

In block 600, the merged medical conversation transcript (from block 504) may be submitted by the workflow worker instance to the sectioning model. In block 602, the results from the sectioning model may be received. An example of results includes the determined sections shown by labeled bracketed sections in the transcript of the medical conversation in FIG. 4. In block 604, the results may be merged with the merged medical conversation transcript so the merged transcript includes the original transcript, the identified medical entities, the identified speaker roles, and the determined sections.

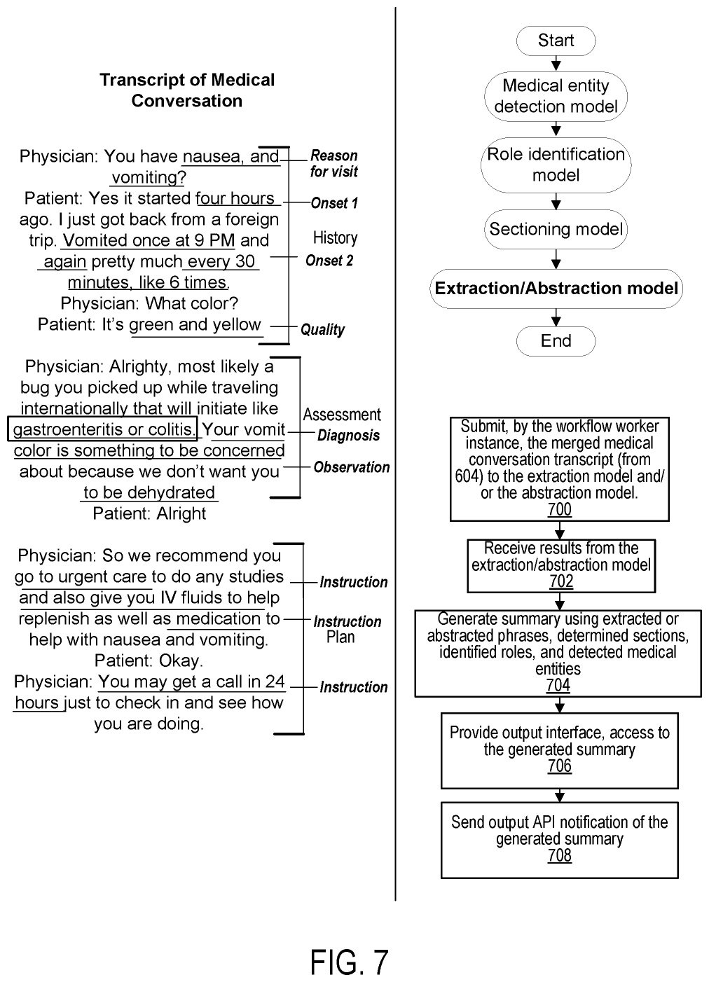

FIG. 7 is a block diagram illustrating extracted phrases in a transcript of a medical conversation, a workflow for generating a summary of the medical conversation, and a process of extracting and/or abstracting phrases from a transcript of the medical conversation, according to some embodiments.

Some embodiments, such as shown in FIGS. 1A, 1B, 2, 3, 4, 5, and 6 may include further features, such as shown in FIG. 7. In some embodiments, a workflow for generating a summary of a transcript for a medical conversation may involve machine learning models including a medical entity detection model, a role identification model, a sectioning model, and an extraction/abstraction model. An engine, such as the work flow processing engine 130 from FIG. 1A, may utilize the extraction/abstraction model to analyze the transcript of the medical conversation and extract phrases for a summary from a transcript as shown in the merged transcript of the medical conversation in FIG. 7. In some embodiments, the extraction/abstraction model may use the extracted phrases to abstract the phrases to generate the summary. In some embodiments, the extraction/abstraction model may skip the extraction step and may analyze the sections of the transcript to abstract phrases from the sections for the summary. For example, a phrase such as “the doctor from urgent care will get you medication to help with nausea and vomiting” may be abstracted to “the doctor will prescribe medication for nausea and vomiting”. In some embodiments, similar phrases may be used in the abstracted phrase to paraphrase phrases in the transcript.

In block 700, the merged medical conversation transcript (from block 604) may be submitted by the workflow worker instance to the extraction/abstraction model. In block 702, the results from the extraction/abstraction model may be received. Results may include important phrases to be included in the generated summary as shown by underlined phrases and short descriptions of the phrases in FIG. 7. In block 704, the summary may be generated using the extracted or abstracted phrases, determined sections, identified roles, and detected medical entities. In block 706, an output interface may be provided access to the generated summary. In block 708, a notification of the generated summary may be sent to an output API.

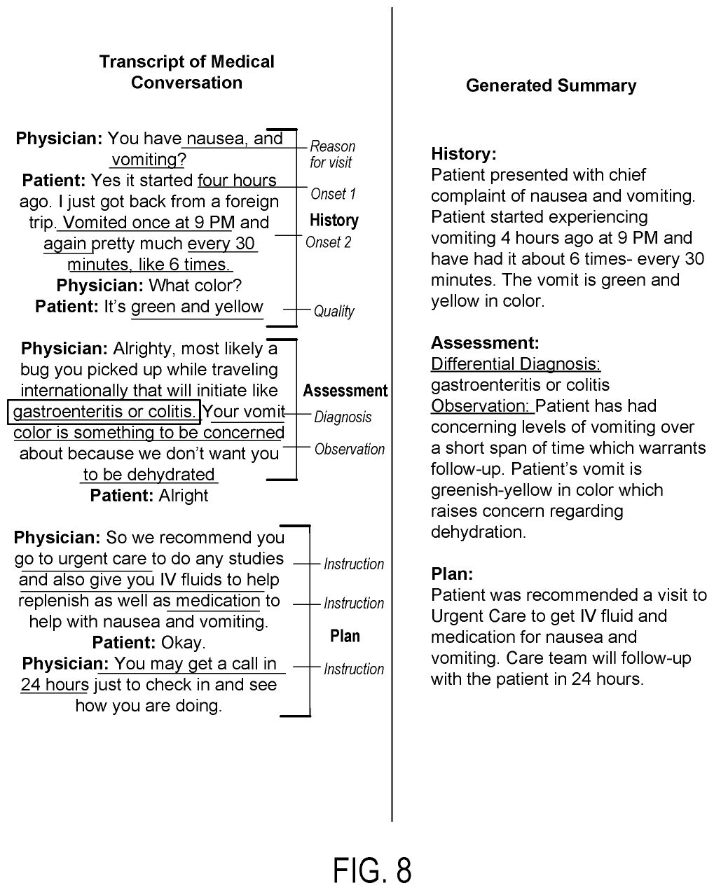

FIG. 8 is a block diagram illustrating an annotated transcript of a medical conversation indicating identified medical entities, identified speaker roles, labeled sections, and extracted phrases of the transcript and a generated summary of the medical conversation that has been generated based on the identified medical entities, the identified speaker roles, the determined sections, and the extracted phrases from the transcript, according to some embodiments.

Some embodiments, such as shown in FIGS. 1A, 1B, 2, 3, 4, 5, 6, and 7 may include further features, such as shown in FIG. 8. An example of a merged transcript of a medical conversation and a generated summary for the transcript may be shown in FIG. 8. In some embodiments, extracted phrases of a section of the merged transcript may be added to the corresponding section of the generated summary, such as shown in FIG. 8. In some embodiments, the generated summary may take phrases from the transcript and paste them into the summary. In some embodiments, the extracted phrases may be reworded into coherent complete sentences for the generated summary using the results of the speaker roles and the medical entities. In some embodiments, the generated summary may include abstracted phrases.

FIG. 9 is a block diagram illustrating four models for generating a summary of a medical conversation, wherein each of the four models are trained by a transcript provided by a preceding model, according to some embodiments.

Some embodiments, such as shown in FIGS. 1A, 1B, 2, 3, 4, 5, 6, 7 and 8 may include further features, such as shown in FIG. 9. Some embodiments for training for machine learning models used in generating a summary of a medical conversation is shown in FIG. 9. In some embodiments, the training may be performed during an offline process. Transcript A 900 may represent an original transcript of a medical conversation and transcript B 902 shows the transcript annotated with medical entities and represents a merged transcript including identified medical entity results. To train a medical entity detection model such as the medical entity detection model 132 in FIG. 1A and the medical entity detection model described in FIG. 4, the medical entity model trainer 904 may use training data including medical conversation transcripts and annotated medical entity transcripts including identified medical entities such as transcript A 900 and transcript B 902, respectively. The medical entity model trainer 904 may output a trained medical entity detection model 906.

Transcript C 908 may represent a transcript annotated with medical entities and speaker roles. To train a role identification model such as the role identification model 134 in FIG. 1A and the role identification model described in FIG. 5, the role identification model trainer 910 may use training data including merged transcripts from the previously trained model such as transcript B 902 and merged transcripts with the role speakers identified such as transcript C 908. The role identification model trainer 910 may output a trained role identification model 912.

Transcript D 914 may represent a transcript annotated with medical entities, speaker roles, and determined sections. To train a sectioning model such as the sectioning model 136 in FIGS. 1A and 1B and the sectioning model described in FIG. 6, the sectioning model trainer 916 may use training data including merged transcripts from the previously trained model such as transcript C 908 and merged transcripts with the determined sections such as transcript D 914. The sectioning model trainer 916 may output a trained sectioning model 918.

Transcript E 920 may represent a transcript annotated with medical entities, speaker roles, determined sections, and extracted phrases. To train an extraction/abstraction model such as the extraction/abstraction model 136 in FIGS. 1A and 1B and the extraction/abstraction model described in FIG. 7, the extraction/abstraction model trainer 922 may use training data including merged transcripts from the previous model such as transcript D 914 and merged transcripts with the extracted phrases such as transcript E 920. The extraction/abstraction model trainer 922 may output a trained extraction model 924. In some embodiments, the extraction/abstraction model may be trained with training data including abstracted phrases from transcripts. In some embodiments the order of training the models may be different. In some embodiments, the machine learning models may be trained using unlabeled training data, such as non-annotated transcripts, wherein each model trainer trains their respective machine learning models using reinforcement learning through confidence scores, or using other suitable techniques for training using un-labeled data. In some embodiments, the training of the machine learning models may be performed independently, wherein the training data may include no merged transcripts.

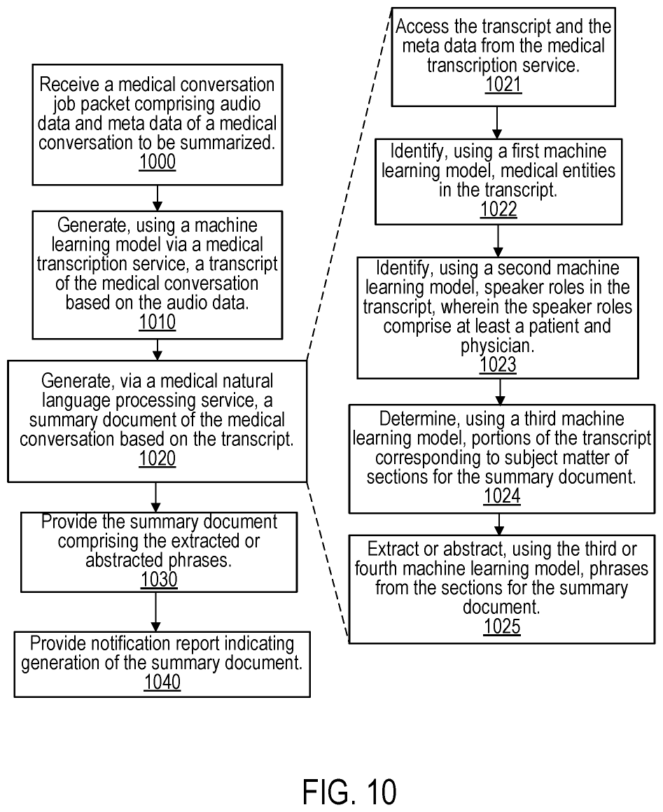

FIG. 10 is a flow diagram illustrating a process of generating a transcript of a medical conversation and generating a summary document of the medical conversation based on the transcript, using four machine learning models, according to some embodiments.

In some embodiments, a process for generating a transcript and a summary of a medical conversation may resemble a process such as that which is shown in FIG. 10. In block 1000, a medical conversation job packet comprising audio data and meta data of a medical conversation to be summarized may be received. In block 1010, a transcript of the medical conversation based on the audio data using a machine learning model via a medical transcription service may be generated. In block 1020, a summary document of the medical conversation based on the transcript may be generated via a medical natural language processing service.

Blocks 1021, 1022, 1023, 1024, and 1025 may further describe block 1020. In block 1021, the transcript and the meta data may be accessed from the medical transcription service. In block 1022, medical entities in the transcript may be identified using a first machine learning model. In block 1023, speaker roles in the transcript, wherein the speaker roles comprise at least a patient and physician may be identified. In block 1024, portions of the transcript corresponding to subject matter of sections for the summary document may be determined using a third machine learning model. In block 1025, phrases may be extracted and/or abstracted, using the third or a fourth machine learning model, from the sections for the summary document. In block 1030, the summary document comprising the extracted or abstracted phrases may be provided. In block 1040, a notification report indicating generation of the summary document may be provided.

FIG. 11 is a block diagram illustrating an example computer system that implements portions of the medical audio summarizing described herein, according to some embodiments.

In at least some embodiments, a server that implements a portion or all of one or more of the technologies described herein, including the techniques for performing medical audio summarizing, may include a general-purpose computer system that includes or is configured to access one or more computer-accessible media. FIG. 11 illustrates such a general-purpose computing device 1100. In the illustrated embodiment, computing device 1100 includes one or more processors 1102 coupled to a system memory 1110 (which may comprise both non-volatile and volatile memory modules) via an input/output (I/O) interface 1108. Computing device 1100 further includes a network interface 1116 coupled to I/O interface 1108.

In various embodiments, computing device 1100 may be a uniprocessor system including one processor 1102, or a multiprocessor system including several processors 1102 (e.g., two, four, eight, or another suitable number). Processors 1102 may be any suitable processors capable of executing instructions. For example, in various embodiments, processors 1102 may be general-purpose or embedded processors implementing any of a variety of instruction set architectures (ISAs), such as the x86, PowerPC, SPARC, or MIPS ISAs, or any other suitable ISA. In multiprocessor systems, each of processors 1102 may commonly, but not necessarily, implement the same ISA. In some implementations, graphics processing units (GPUs) may be used instead of, or in addition to, conventional processors.

System memory 1110 may be configured to store instructions and data accessible by processor(s) 1102. In at least some embodiments, the system memory 1110 may comprise both volatile and non-volatile portions; in other embodiments, only volatile memory may be used. In various embodiments, the volatile portion of system memory 1110 may be implemented using any suitable memory technology, such as static random-access memory (SRAM), synchronous dynamic RAM or any other type of memory. For the non-volatile portion of system memory (which may comprise one or more NVDIMMs, for example), in some embodiments flash-based memory devices, including NAND-flash devices, may be used. In at least some embodiments, the non-volatile portion of the system memory may include a power source, such as a supercapacitor or other power storage device (e.g., a battery).

In various embodiments, memristor based resistive random-access memory (ReRAM), three-dimensional NAND technologies, Ferroelectric RAM, magnetoresistive RAM (MRAM), or any of various types of phase change memory (PCM) may be used at least for the non-volatile portion of system memory. In the illustrated embodiment, program instructions and data implementing one or more desired functions, such as those methods, techniques, and data described above, are shown stored within system memory 1110 as program instructions for medical audio summarizing 1112 and medical audio summarizing data 1114. For example, program instructions for medical audio summarizing 1112 may include program instructions for implementing a medical audio summarization service, such as medical audio summarization service 100 illustrated in FIG. 1A. Also, in some embodiments, program instructions for medical audio summarizing 1112 may include program instructions for implementing components of a medical audio summarizing service, such as input interface 102, audio storage 104, meta data managing system 106, customer interface 108, medical transcription engine 110, medical natural language processing engine 122, etc.

In one embodiment, I/O interface 1108 may be configured to coordinate I/O traffic between processor 1102, system memory 1110, and any peripheral devices in the device, including network interface 1116 or other peripheral interfaces such as various types of persistent and/or volatile storage devices. In some embodiments, I/O interface 1108 may perform any necessary protocol, timing, or other data transformations to convert data signals from one component (e.g., system memory 1110) into a format suitable for use by another component (e.g., processor 1102).

In some embodiments, I/O interface 1108 may include support for devices attached through various types of peripheral buses, such as a variant of the Peripheral Component Interconnect (PCI) bus standard or the Universal Serial Bus (USB) standard, for example. In some embodiments, the function of I/O interface 1108 may be split into two or more separate components, such as a north bridge and a south bridge, for example. Also, in some embodiments some or all of the functionality of I/O interface 1108, such as an interface to system memory 1110, may be incorporated directly into processor 1102.

Network interface 1116 may be configured to allow data to be exchanged between computing device 1100 and other devices 1120 attached to a network or networks 1118, such as other computer systems or devices as illustrated in FIG. 1A through FIG. 10, for example. Additionally, network interface 1116 may support communication via telecommunications/telephony networks such as analog voice networks or digital fiber communications networks, via storage area networks such as Fibre Channel SANs, or via any other suitable type of network and/or protocol.

In some embodiments, system memory 1110 may be one embodiment of a computer-accessible medium configured to store program instructions and data as described above for FIG. 1A through FIG. 10 for implementing embodiments of the corresponding methods and apparatus. However, in other embodiments, program instructions and/or data may be received, sent, or stored upon different types of computer-accessible media. Generally speaking, a computer-accessible medium may include non-transitory storage media or memory media such as magnetic or optical media, e.g., disk or DVD/CD coupled to computing device 1100 via I/O interface 1108. A non-transitory computer-accessible storage medium may also include any volatile or non-volatile media such as RAM (e.g., SDRAM, DDR SDRAM, RDRAM, SRAM, etc.), ROM, etc., that may be included in some embodiments of computing device 1100 as system memory 1110 or another type of memory.

In some embodiments, a plurality of non-transitory computer-readable storage media may collectively store program instructions that when executed on or across one or more processors implement at least a subset of the methods and techniques described above. A computer-accessible medium may include transmission media or signals such as electrical, electromagnetic, or digital signals, conveyed via a communication medium such as a network and/or a wireless link, such as may be implemented via network interface 1116.

Portions or all of multiple computing devices such as that illustrated in FIG. 11 may be used to implement the described functionality in various embodiments; for example, software components running on a variety of different devices and servers may collaborate to provide the functionality. In some embodiments, portions of the described functionality may be implemented using storage devices, network devices, or special-purpose computer systems, in addition to or instead of being implemented using general-purpose computer systems. The term “computing device,” as used herein, refers to at least all these types of devices, and is not limited to these types of devices.

The various methods as illustrated in the figures and described herein represent example embodiments of methods. The methods may be implemented in software, hardware, or a combination thereof. The order of method may be changed, and various elements may be added, reordered, combined, omitted, modified, etc.

Various modifications and changes may be made as would be obvious to a person skilled in the art having the benefit of this disclosure. It is intended that the invention encompasses all such modifications and changes and, accordingly, the above description to be regarded in an illustrative rather than a restrictive sense.

What is claimed is:

1. A system comprising:
one or more computing devices configured to:
receive a medical conversation job packet to be summarized, comprising audio data of a medical conversation and meta data for the medical conversation;
generate, via a medical transcription service, a transcript of the medical conversation based on the audio data of the medical conversation; and
generate, via a medical natural language processing service, a summary document of the medical conversation based on the transcript,
wherein to generate the summary document, the medical natural language processing service is configured to:
access the transcript and the meta data from the medical transcription service;
identify, using a first machine learning model, medical entities in the transcript;
identify, using a second machine learning model, speaker roles in the transcript, wherein the speaker roles comprise at least a patient and a physician;
determine, using a third machine learning model, portions of the transcript corresponding to subject matter of sections for the summary document;
extract or abstract, using the third machine learning model or a fourth machine learning model, phrases from each of the sections for the summary document; and
provide the summary document comprising the subject matter of the extracted or abstracted phrases included in the corresponding sections,
wherein at least one or more of the first, second, third, or fourth machine learning models are provided merged transcripts comprising results from one or more ones of the preceding machine learning models.

2. The system of claim 1, wherein:
the first machine learning model is a medical entity detection model, wherein the medical entity detection model has been trained using transcripts comprising medical entities;
the second machine learning model is a role identification model, wherein the role identification model has been trained using annotated transcripts indicating physician and patient roles along with identified medical entities determined using the first machine learning model;
the third machine learning model is a sectioning model, wherein the sectioning model has been trained using transcripts comprising labeled sections along with identified medical entities determined using the first machine learning model and identified roles determined using the second machine learning model; and
the fourth machine learning model is an extraction model or an abstraction model, wherein the extraction model or an abstraction model has been trained using annotated transcripts indicating phrases to be included in respective summaries along with medical entities determined using the first machine learning model, identified roles determined using the second machine learning model, and identified sections determined using the third machine learning model.

3. The system of claim 1, wherein to generate the transcript of the medical conversation, the medical transcription service is configured to use an additional machine learning model for the transcription generation, wherein the additional machine learning model has been trained using audio training data.

4. The system of claim 1, the one or more computing devices are configured to implement an application programmatic interface (API) for providing the summary document for upload to an electronic health record.

5. A method comprising:
receiving a transcript of a medical conversation and meta data for the medical conversation;
generating, via a medical natural language processing service, a summary document of the medical conversation based on the transcript,
wherein said generating the summary document, via the medical natural language processing service, comprises:
accessing the transcript and the meta data from the medical transcription service;
identifying, using a first machine learning model, medical entities in the transcript;
identifying, using a second machine learning model, speaker roles in the transcript, wherein the speaker roles comprise at least a patient and a physician;
determining, using a third machine learning model, portions of the transcript corresponding to subject matter of sections for the summary document;
extracting or abstracting, using the third machine learning model or a fourth machine learning model, phrases from each of the sections for the summary document; and
providing the summary document comprising the subject matter of the extracted or abstracted phrases included in the corresponding sections,
wherein at least one or more of the first, second, third, or fourth machine learning models are provided merged transcripts comprising results from one or more ones of the preceding machine learning models.

6. The method of claim 5, wherein the first machine learning model is a medical entity detection model, wherein the medical entity detection model has been trained using transcripts comprising medical entities.

7. The method of claim 5, wherein the second machine learning model is a role identification model, wherein the role identification model has been trained using annotated transcripts indicating physician and patient roles.

8. The method of claim 5, wherein a summarization module comprises the third machine learning model and the fourth machine learning model, wherein the summarization module is used to summarize the transcript.

9. The method of claim 8, wherein the third machine learning model is a sectioning model, wherein the sectioning model has been trained using transcripts comprising labeled sections.

10. The method of claim 9, wherein the fourth machine learning model is an extraction model and an abstraction model, wherein the extraction model and the abstraction model has been trained using annotated transcripts indicating phrases to be included in respective summaries.

11. The method of claim 10, wherein to generate the summary document the medical natural language processing service is configured to use the summarization module to perform summarization based on outputs from the sectioning model, the extraction model, and the abstraction model.

12. The method of claim 10, wherein to generate the summary document the medical natural language processing service is configured to use the summarization module to perform summarization based on outputs from the sectioning model and the abstraction model.

13. The method of claim 11, further comprising:
receiving customer report preferences; and
updating the summarization module based on the customer report preferences.

14. The method of claim 5, comprising:
receiving customer supplied training data, via an interface; and
determining a format to be used for the summary document based on the customer supplied training data.

15. The method of claim 14, comprising:
updating, using the customer supplied training data, a given one of the first, second, third, or fourth machine learning models.

16. The method of claim 5, comprising:
receiving a medical conversation job packet to be summarized, comprising audio data of the medical conversation and meta data for the medical conversation; and
generating, via a medical transcription service, the transcript of the medical conversation based on the audio data of the medical conversation.

17. The method of claim 16, comprising:
receiving customer supplied training data, via an interface;
updating, using the customer supplied training data, the transcription service model.

18. A non-transitory, computer-readable medium storing program instructions that, when executed using one or more processors, cause the one or more processors to:
receive a transcript of a medical conversation and meta data for the medical conversation;
generate, via a medical natural language processing service, a summary document of the medical conversation based on the transcript,
wherein to generate the summary document, the medical natural language processing service is configured to:
access the transcript and the meta data from the medical transcription service;
identify, using a first machine learning model, medical entities in the transcript;
identify, using a second machine learning model, speaker roles in the transcript, wherein the speaker roles comprise at least a patient and a physician;
determine, using a third machine learning model, portions of the transcript corresponding to subject matter of sections for the summary document;
extract or abstract, using the third machine learning model or a fourth machine learning model, phrases from each of the sections for the summary document; and
provide the summary document comprising the subject matter of the extracted or abstracted phrases included in the corresponding sections,
wherein at least one or more of the first, second, third, or fourth machine learning models are provided merged transcripts comprising results from one or more ones of the preceding machine learning models.

19. The non-transitory, computer-readable medium storing program instructions of claim 18, wherein the second machine learning model is a role identification model, wherein the role identification model has been trained using annotated transcripts indicating physician and patient roles.

20. The non-transitory, computer-readable medium storing program instructions of claim 18, wherein the programming instructions when executed on or across the one or more processors cause the one or more processors to:
receive a first amount of the audio data of the medical conversation and the meta data for the medical conversation;
generate, via a medical transcription service, a transcript of the medical conversation based on the audio data of the medical conversation; and
receive a second amount of the audio data of the medical conversation while continuing to generate the transcript, wherein the transcript is for the first and second amount of the audio data.
